(19) United States
(12) Patent Application Publication
(10) Pub. No.: US 2007/0010426 A1
(43) Pub. Date: Jan. 11, 2007

(54) Composition for preventing and treating cardiovascular disorders

(75) Inventors: Frank Chiahung Mao, Taichung City (TW); Wen-Ying Chen, Longjing Township (TW); Yi-Chung Chiang, Taipei City (TW); Ling-Hui Cheng Chiang, Taipei City (TW)

Correspondence Address: BACON & THOMAS, PLLC, 625 SLATERS LANE, FOURTH FLOOR, ALEXANDRIA, VA, 22314, US

(73) Assignee: Maxluck Biotechnology Corp., Taipei City (TW)

(21) Appl. No.: 11/218,642

(22) Filed: Sep. 6, 2005

(30) Foreign Application Priority Data
Jul. 5, 2005 (TW) 094122677

(51) Int. Cl. A61K 38/40

(52) U.S. Cl. 514/6

(57) ABSTRACT

A composition for preventing and treating cardiovascular disorders is disclosed. The composition includes the lactoferrin and a trivalent chromium compound. The trivalent chromium compound of the present invention is selected from a group consisting of chromium (III) chloride hexahydrate, chromium (III) chloride, chromium (III) acetate, chromium (III) sulfate, chromium picolinate, chromium nicotinate, inorganic salts of trivalent chromium, organic salts of trivalent chromium, and combinations thereof. The present invention also discloses a method for preventing and treating cardiovascular disease of an acceptor.

### BACKGROUND OF THE INVENTION

1. Field of the Invention

The present invention relates to a composition and method for preventing and treating cardiovascular disorders and, more particularly, to a trivalent chromium dairy product that can prevent and treat cardiovascular disease of an acceptor and the manufacturing method thereof.

2. Description of the Related Art

Owing to the development in economics, the change in lifestyle, and the abundance of rich foods, obesity is gradually found in all age groups of modern humans, from the children, the youth, to the aged people. The population of obese people keeps increasing and, accordingly, there are more and more people suffering from the derivative sicknesses of obesity, such as hypertension, heart disease, and hyperlipidemia. Therefore, it is really important for the modern humans to study how to prevent and treat cardiovascular disease.

Normally, the trivalent chromium absorbed from foods can be transferred into glucose tolerance factor (GTF) and then distributed in the tissues of human bodies. GTF in the tissues assists blood lipids and hydrocarbons in undergoing normal metabolism through the synergistic effect with insulin.

It is revealed from the research that the concentration of serum chromium decreases as one gets older. From the clinical research in 1997, Davies verified that the concentration of serum chromium decreases from 0.5 ng/ml at one's childhood to 0.3 ng/ml at the age of 70. Obesity is a cause that drains chromium from a human body. Moreover, the deficiency of chromium will lead to problems in metabolism of myocardial cells, which subsequently causes myocardial infarction and other clinical symptoms.

Chromium may be absorbed in the forms of inorganic salt or organic salt from the daily food. However, the absorption rate of inorganic chromium for human body is very low, and only ranges from 0.4% to 3%. The root cause lies in that the inorganic chromium tends to undergo olation reaction in the digestive tract. The olation reaction may produce bulky complex compounds that hinder the intestine tract from absorption.

The adequate organic chromium includes chromium picolinate, chromium nicotinate, chromium GTF (Glucose Tolerance Factor), and chromium yeast extract.

Organic chromium supplement helps to remedy the cardiovascular disease caused by the shortage of chromium. For the general adults, chromium combined with other kinds of vitamins and mineral substances may be deemed as a personal nutriment supplement.

U.S. Pat. No. 4,923,855 disclosed a synthetic GTF chromium material and process therefore, in which the trivalent chromium is combined with nicotinic acid to obtain a novel chromium product having a glucose tolerance factor. In 2002, Cefalu et al. announced that chromium picolinate could reduce the blood lipids of an obese mouse.

### SUMMARY OF THE INVENTION

The present invention provides a composition for preventing and treating cardiovascular disorders. More particularly, the present invention provides a composition of trivalent chromium compound and lactoferrin that can prevent and treat cardiovascular disorders. The present invention also provides a method for preventing and treating cardiovascular disorders of an acceptor. The method administrates an effective amount of a composition that prevents and treats cardiovascular disorders to the acceptor. The composition is composed of trivalent chromium compound and lactoferrin.

The composition for preventing and treating cardiovascular disorders of the present invention mainly includes (a) a lactoferrin and (b) a trivalent chromium compound.

The lactoferrin of the present invention is not restricted, and can come from cow milk lactoferrin, goat milk lactoferrin, unpurified cow milk, and unpurified goat milk. Because lactoferrin mainly exists in the whey of the milk, the lactoferrin of the present invention can also be replaced with whey protein products or milk products.

The trivalent chromium compound of the present invention is not restricted, either. Preferably, it can be selected from a group consisting of chromium (III) chloride hexahydrate, chromium (III) chloride, chromium (III) acetate, chromium (III) sulfate, chromium picolinate, chromium nicotinate, inorganic salts of trivalent chromium, organic salts of trivalent chromium, and combinations thereof.

The inorganic salt of trivalent chromium includes, for example, chromium (III) chloride and chromium (III) sulfate.

The organic salt of trivalent chromium includes, for example, chromium (III) acetate, chromium picolinate, chromium nicotinate, amino acid chelated chromium, chromium yeast extract, and chromium yeast.

More preferably, the trivalent chromium compound is chromium (III) chloride hexahydrate, chromium (III) chloride, chromium (III) acetate, chromium (III) sulfate, chromium picolinate, or chromium nicotinate.

Generally speaking, the molar ratio of lactoferrin to the trivalent chromium compound of the present invention is not particularly restricted. Preferably, the molar ratio of lactoferrin to the trivalent chromium compound ranges from 1:200 to 10:1. More preferably, the molar ratio of lactoferrin to the trivalent chromium compound ranges from 1:20 to 1:1.

The composition of the present invention can serve as an additive of a dairy product. The dairy product can be the fresh milk of mammals, long-life milk, concentrated milk, cheese, or milk powder.

The composition containing trivalent chromium lactoferrin of the present invention can be absorbed and utilized effectively by the human body. Taking the dairy product having the composition of the present invention, not only can it replenish the organic chromium efficiently, it also can control the level of blood lipids and inflammation factors of a patient suffering from cardiovascular disease.

The composition containing trivalent chromium lactoferrin of the present invention is formed by mixing the trivalent chromium compound with the lactoferrin, and can enhance the normal metabolism of fat, carbohydrates, and protein. The lactoferrin is a glycoprotein that is capable of combining with metal ions. Each lactoferrin molecule can be combined with two trivalent chromium ions.

The composition of the present invention can be used to form a medicine. Also, it can be added into a dairy product, and thereby form a dairy product containing trivalent chromium compound and lactoferrin, i.e., form a food or nutriment.

The composition of the present invention can be taken by a patient suffering from cardiovascular disease because the composition can supplement the trivalent chromium effectively and enhance the normal metabolism of fat, carbohydrates, and protein. In addition, the level of blood lipids and inflammation factors can be reduced to comfort the sufferers of cardiovascular disease.

### DETAILED DESCRIPTION OF THE PREFERRED EMBODIMENTS

The composition of the present invention can be formed by mixing the powder of lactoferrin with the powder of trivalent chromium compound. Moreover, water can also be added into the mixture of lactoferrin and the trivalent chromium compound to form a mixed solution. The mixed solution can be heated properly so that the mixing can be done adequately. The heating temperature ranges around 37° C. to 95° C., and preferably ranges from 50° C. to 80° C. The well-mixed solution is then spray-dried to form the composition containing trivalent chromium lactoferrin of the present invention.

The raw material of trivalent chromium compound used in the present invention can be the form of inorganic salt or organic salt, such as chromium (III) chloride hexahydrate, chromium (III) chloride, chromium (III) acetate, chromium (III) sulfate, chromium picolinate, and chromium nicotinate.

Lactoferrin could come from the solution or dry powder of lactoferrin. Because lactoferrin mainly exists in the whey of the milk, the present invention can also use an unpurified whey protein product or a dairy product to replace lactoferrin.

The following detailed description is given by way of example and not intended to limit the invention solely to the embodiments described herein.

### EXAMPLE 1

Mix 5 g of lactoferrin powder with 0.5 g of chromium (III) chloride hexahydrate to form the composition containing trivalent chromium lactoferrin of the present invention.

### EXAMPLE 2

Mix 5 g of lactoferrin powder and 0.5 g of chromium (III) chloride hexahydrate with 1 liter of water to form a solution. The solution is well-mixed and then spray-dried to form the composition containing trivalent chromium lactoferrin of the present invention.

### EXAMPLE 3

Mix 5 g of lactoferrin powder and 0.5 g of chromium (III) chloride hexahydrate with 1 liter of water to form a solution. The solution is well-mixed, spray-dried, and then mixed with 10 kg of milk powder to form the dairy product containing trivalent chromium lactoferrin.

### EXAMPLE 4

Mix 100 g of whey protein and 0.5 g of chromium (III) chloride hexahydrate with 3 liters of water to form a solution. The solution is well-mixed and then spray-dried to form the composition containing trivalent chromium lactoferrin of the present invention.

### EXAMPLE 5

The procedure of Example 4 is repeated first, and then the product is mixed with 10 kg of milk powder to form the dairy product containing trivalent chromium lactoferrin.

### EXAMPLE 6

The procedure of Example 4 is repeated, except that the mixed solution is added into 90 kg of fresh milk to form the dairy product containing trivalent chromium lactoferrin.

### EXAMPLE 7

Mix 5 g of lactoferrin powder with 0.3 g of chromium (III) chloride to form the composition containing trivalent chromium lactoferrin of the present invention.

### EXAMPLE 8

Mix 6 g of lactoferrin powder with 0.5 g of chromium acetate to form the composition containing trivalent chromium lactoferrin of the present invention.

### EXAMPLE 9

Mix 5 g of lactoferrin powder and 0.35 g of chromium sulfate with 1 liter of water to form a solution. The solution is well-mixed and then spray-dried to form the composition containing trivalent chromium lactoferrin of the present invention.

### EXAMPLE 10

Mix 5 g of lactoferrin powder and 0.8 g of chromium picolinate with 1 liter of water to form a solution. The solution is heated and mixed, and then spray-dried to form the composition containing trivalent chromium lactoferrin of the present invention.

### EXAMPLE 11

Mix 5 g of lactoferrin powder and 0.8 g of chromium nicotinate with 1 liter of water to form a solution. The solution is heated and mixed, and then spray-dried to form the composition containing trivalent chromium lactoferrin of the present invention.

### TEST EXAMPLE 1

The dairy product obtained from Example 5 is mixed into a mouse diet (Modified LabDiet w/35.5% Lard, PMI® Richmond, Ind., USA). At 13 weeks of age, glucose tolerance tests (GTT) are performed after an overnight fast. According to the results of GTT, the KK/HlJ mice are randomly divided into two groups. The experimental group is supplied with a dairy product containing 800 ppb per day of trivalent chromium, the control group is not supplied. After 5 weeks of chromium supplementation, the KK/HlJ mice are fasted overnight and GTT is performed again. The changes of blood glucose during the GTT are shown in Table 1. At the beginning of the experiment, the levels of blood glucose are similar in two groups. However, the levels of blood glucose at 30, 60, 120 and 180 minutes are significantly reduced in the experimental group after receiving a chromium supplementation for 5 weeks. These results suggest that glucose tolerance is significantly improved in the experimental group.

TABLE 1

| | Control group | Experimental group |
|---|---|---|
| Baseline | (N = 12) | (N = 12) |
| 0 min | 163 ± 30 | 166 ± 20 |
| 30 min | 335 ± 46 | 334 ± 24 |
| 60 min | 365 ± 61 | 356 ± 36 |
| 120 min | 287 ± 67 | 267 ± 67 |
| 180 min | 215 ± 66 | 196 ± 61 |
| Curve area | 876 ± 147 | 841 ± 117 |
| After supplement | Control group | Experimental |
| for 5 weeks | (N = 12) | (N = 12) |
| 0 min | 162 ± 30 | 141 ± 21 |
| 30 min | 311 ± 29 | 275 ± 35* |
| 60 min | 383 ± 30 | 287 ± 41*** |
| 120 min | 338 ± 64 | 210 ± 62*** |
| 180 min | 246 ± 64 | 168 ± 67* |
| Curve area | 944 ± 104 | 682 ± 129*** |

*p < 0.05, significant difference vs. control group.
**p < 0.01, significant difference vs. control group.

### TEST EXAMPLE 2

The dairy product obtained from Example 5 is mixed into the mouse diet (Modified LabDiet w/35.5% Lard, PMI® Richmond, Ind., USA). At 13 weeks of age, glucose tolerance tests (GTT) are performed after an overnight fast. According to the results of GTT, the KK/HlJ mice are randomly divided into two groups. The experimental group is supplied with a dairy product containing 800 ppb per day of trivalent chromium, the control group is not supplied. After 7 weeks of chromium supplementation, the KK/HlJ mice are fasted overnight and the levels of blood glucose, triglycerides, LDL-C (low-density lipoprotein cholesterol), insulin, leptin and interlukin-6 (IL-6) are analyzed. The results are shown below in Table 2. As a result, the levels of blood glucose, triglycerides, LDL-C, insulin, leptin and IL-6 are significantly reduced in the serum of KK/HlJ mice supplied with a chromium dairy product compared to that of the control KK/HlJ mice. These results show that dyslipidemia, hyperinsulinemia and hyperleptinemia are significantly improved and an inflammatory marker is significantly reduced in the experimental group. Therefore, these results suggest that the chromium dairy product is beneficial in the reducing of cardiovascular risk markers and can further prevent and treat cardiovascular disease.

TABLE 2

| | Control group | Experimental group |
|---|---|---|
| Blood glucose (mg/dl) | 173 ± 30 | 131 ± 13* |
| Triglycerides (mg/dl) | 166 ± 35 | 123 ± 13* |
| Total cholesterol | 145 ± 23 | 150 ± 14 |
| (mg/dl) | | |
| LDL (mg/dl) | 10.7 ± 2.2 | 7.8 ± 1.5* |
| Insulin (ng/ml) | 2.6 ± 1.0 | 0.7 ± 0.4* |
| Leptin (ng/ml) | 28 ± 10 | 18 ± 7* |
| IL-6 (pg/ml) | 32 ± 16 | 18 ± 15* |

*p < 0.05, significant difference vs. control group.

### TEST EXAMPLE 3

The dairy product obtained from Example 5 is mixed into mouse diet (Modified LabDiet w/35.5% Lard, PMI® Richmond, Ind., USA). At 13 weeks of age, glucose tolerance tests (GTT) are performed after an overnight fast. According to the results of GTT, the KK/HlJ mice are randomly divided into two groups. The experimental group is supplied with a dairy product containing 800 ppb per day of trivalent chromium, the control group is not supplied. After 7 weeks of chromium supply, the KK/H1J mice are sacrificed and their hearts are harvested. The heart infarction size is evaluated by triphenyltetrazolium chloride (TTC) staining. Infarction sizes are revealed on the outer border of hearts in the control mice. Supplementing the dairy product with chromium and providing it to the experimental mice significantly reduces the myocardial infarction size when compared to the control group (p<0.05).

TABLE 3

| Control group | Experimental group |
|---|---|
| Myocardial infarction size | Myocardial infarction size |
| 16.8 ± 7.3% | 3.1 ± 3.5%* |

*p < 0.05, significant difference vs. control group.

### TEST EXAMPLE 4

The dairy product obtained from Example 5 is mixed into the mouse diet (Modified LabDiet w/35.5% Lard, PMI®( Richmond, Ind., USA). At 13 weeks of age, glucose tolerance tests (GTT) are performed after an overnight fast. According to the results of GTT, the KK/HlJ mice are randomly divided into two groups. The experimental group is supplied with milk powder containing 800 ppb per day of trivalent chromium, the control group is not supplied. After 7 weeks of chromium supplementation to the diet, the KK/H1J mice are sacrificed and their hearts are harvested. Then, the hearts are fixed with 10% neutral formalin solution and embedded with paraffin wax. Serial sections (10 μm thick) are cut from each specimen and stained with hematoxylin and eosin (H&E). After staining, the sections are analyzed under light microscopy. Necrosis areas on the outer border of myocardium are observed in the control mice. High magnification reveals myocyte degeneration and macrophage infiltration in the necrosis area. However, the necrosis areas are significantly reduced and myocytes are normal in the experimental mice. These results suggest that chromium dairy product supplementation improves myocardial necrosis in the experimental mice.

The composition containing trivalent chromium lactoferrin of the present invention can be taken by a patient suffering from cardiovascular disease because it can reduce cardiovascular risk markers thereof effectively. From Table 1, Table 2, and Table 3, it is proven that the cardiovascular disorders are improved effectively after the dairy product containing the composition of the present invention is taken.

Although the present invention has been explained in relation to its preferred embodiments, it is to be understood that many other possible modifications and variations can be made without departing from the scope of the invention as hereinafter claimed.

What is claimed is:

1. A composition for preventing and treating cardiovascular disorders, comprising:
a lactoferrin; and
a trivalent chromium compound;
wherein the trivalent chromium compound is selected from the group consisting of chromium (III) chloride hexahydrate, chromium (III) chloride, chromium (III) acetate, chromium (III) sulfate, chromium picolinate, chromium nicotinate, inorganic salts of trivalent chromium, organic salts of trivalent chromium, and combinations thereof.

2. The composition as claimed in claim 1, wherein the molar ratio of the lactoferrin to the trivalent chromium compound ranges from 1:200 to 10:1.

3. The composition as claimed in claim 1, wherein the molar ratio of the lactoferrin to the trivalent chromium compound ranges from 1:20 to 1:1.

4. The composition as claimed in claim 1, wherein the lactoferrin comes from unpurified milk or whey protein.

5. The composition as claimed in claim 1, wherein the lactoferrin comes from the group consisting of cow milk lactoferrin, goat milk lactoferrin, unpurified cow milk, unpurified goat milk, and combinations thereof.

6. The composition as claimed in claim 1, wherein the trivalent chromium compound is selected from the group consisting of chromium (III) chloride hexahydrate, chromium (III) chloride, chromium (III) acetate, chromium (III) sulfate, chromium picolinate, chromium nicotinate, and combinations thereof.

7. The composition as claimed in claim 1, wherein the composition serves as an additive of a dairy product, which is selected from a group consisting of fresh milk of mammals, long-life milk, concentrated milk, fermented milk, cheese, and milk powder.

8. A method for preventing and treating cardiovascular disorders of an acceptor, comprising administrating an effective amount of a composition for preventing and treating cardiovascular disorders to the acceptor, wherein the composition comprises:
a lactoferrin; and
a trivalent chromium compound;
wherein the trivalent chromium compound is selected from the group consisting of chromium (III) chloride hexahydrate, chromium (III) chloride, chromium (III) acetate, chromium (III) sulfate, chromium picolinate, chromium nicotinate, inorganic salts of trivalent chromium, organic salts of trivalent chromium, and combinations thereof.

9. The method as claimed in claim 8, wherein the molar ratio of the lactoferrin to the trivalent chromium compound ranges from 1:200 to 10:1.

10. The method as claimed in claim 8, wherein the molar ratio of the lactoferrin to the trivalent chromium compound ranges from 1:20 to 1:1.

11. The method as claimed in claim 8, wherein the lactoferrin comes from unpurified milk or whey protein.

12. The method as claimed in claim 8, wherein the lactoferrin comes from a group consisting of cow milk lactoferrin, goat milk lactoferrin, unpurified cow milk, and unpurified goat milk.

13. The method as claimed in claim 8, wherein the trivalent chromium compound is selected from a group consisting of chromium (III) chloride hexahydrate, chromium (III) chloride, chromium (III) acetate, chromium (III) sulfate, chromium picolinate, and chromium nicotinate.

14. The method as claimed in claim 8, wherein the composition serves as an additive of a dairy product, which is selected from a group consisting of fresh milk of mammals, long-life milk, concentrated milk, fermented milk, cheese, and milk powder.
